(19) United States
(12) Patent Application Publication
(10) Pub. No.: US 2005/0221322 A1
(43) Pub. Date: Oct. 6, 2005

(54) Multiple closed nucleus breeding for swine production

(76) Inventor: James David Fox, Sycamore, IL (US)

Correspondence Address: HOWREY LLP, C/O IP DOCKETING DEPARTMENT, 2941 FAIRVIEW PARK DRIVE SUITE 200, FALLS CHURCH, VA, 22042, US

(21) Appl. No.: 10/512,900

(22) PCT Filed: May 12, 2003
(86) PCT No.: PCT/US03/14998

§ 371 Date: Oct. 28, 2004

Related U.S. Application Data
(60) Provisional application No. 60/380,786, filed on May 14, 2002.

(51) Int. Cl. C12Q 1/68; A01K 67/027

(52) U.S. Cl. 435/6; 800/17

(57) ABSTRACT

The invention comprises use of first and second, and optionally additional further, genetically and information-linked swine nucleus breeding herds to transmit genetic improvement from the first herd to each of the other herds which can be closed to live animal introduction to provide benefits in addition to genetic improvement to both the first and second and any additional herds.

### FIELD OF THE INVENTION

The invention relates to the production of swine and in particular aspects to methods and systems using two or more nucleus herds for breeding and delivery of improved genetics with health to swine producers. In one aspect, the invention relates to such methods and systems in which two genetically linked nucleus herds are used cooperatively to improve genetics in one or both herds. In other aspects, the invention relates to such methods and systems comprising more than two nucleus herds.

### BACKGROUND OF THE INVENTION

Animal breeding and production has changed significantly in recent decades and ongoing concerns about animal health and transmissible diseases will cause change to continue in the direction of increasing herd health. Further, as breeding has increasingly focused on polygenic and other low response traits, statistics and technology have come to play an increasing role. One result of this trend has been the development and widespread use of the best linear unbiased prediction (BLUP) statistical model for predicting estimated breeding values (EBVs) for potential parents to be used in a breeding program. Such programs are well known and available to persons skilled in the art from a number of sources and are capable of producing EBVs that to a considerable extent distinguish between genetic and environmental effects. BLUP programs for use with swine are well known and available. One system that can provide advantageous results can be mentioned here, but other programs are available and the approach is well known to those skilled in the art of animal breeding. The MTDFREML (Multiple Trait Derivative Free Restricted Maximum Likelihood) is a flexible set of programs, designed to be used with animal breeding data where an animal genetic effect is used for each trait, that can be used to estimate variance components using a derivative free restricted maximum likelihood algorithm. The programs are readily available. The program generates BLUP solutions to the mixed model equations, contrasts of solutions, prediction error variances of solutions and contrasts, and calculates expected values of solutions. The programs are readily available from Dale Van Vleck at the University of Nebraska—Lincoln. As will be familiar to those working in this area, some development of input and output routines may require development for particular applications, but these are matters involving only the routine exercise of ordinary skill.

Since it is impractical to include all animals in the swine industry in a single breeding program because of costs and lack of control, genetic improvement of swine breeding stock has tended to involve use of relatively few elite breeding units or genetic nucleus herds, for example, at the top of pyramid structures to disseminate genetic improvement to terminal swine produced for food. Open and closed nucleus pyramid breeding schemes are now extensively used to provide an advantageous balance between rate of genetic improvement and rate of inbreeding in producing breeding stock for terminal swine (non-breeding swine used for meat). The difference between open and closed nucleus herds is that in the closed system, the elite breeding herd is closed to the importation of animals from other sources, whereas in open systems the elite breeding herd is open to such animals.

Genetic improvement is strongly dependent on abundant and good measurement of phenotypic traits to be included or excluded for breeding and these measurements are a significant part of the cost and a major contributor to the accuracy of a breeding program. The phenotypic measurements can be turned into EBVs (Estimated Breeding Values), either directly or by including phenotypic data collected from animals across herds in different environments, so that genetic and environmental influences on the data can be distinguished. If data are available having good data structure (use of breeding animals across herds) and proper pedigree recording, BLUP can be advantageously used. Currently, systems for applying BLUP to molecular genetic markers as well as to phenotypic measurements are in preliminary use and under development and it is expected that these techniques will contribute to further improve swine breeding.

In addition to good measurements and good data structure, sophisticated pyramid breeding systems require nucleus herds of sufficiently large size to identify and track phenotypic and genetic markers of interest for the desired genetic improvement. For traits having low heritabilities or showing low levels of improvement per generation, even larger herds are needed to produce statistically meaningful results. These factors and others lead to the requirement of SGNs (swine genetic nucleus herds) of significant size to prevent inbreeding and be capable of reliable genetic improvement in respect of all of the traits of current interest. Since the cost of maintaining and improving large SGNs has been generally prohibitive for the producer of terminal swine, the SGNs have typically been maintained at facilities of commercial genetics suppliers who then distribute animals and semen to producers for use in producing dams and sires for breeding and cross-breeding and ultimately producing terminal swine for the meat markets. As a result, however, the terminal swine producer has lost a measure of direct control over its own breeding program and, as live animals are periodically introduced into its herds, suffers the risk of pathogen importation as well.

As will be described below in more detail, the invention is directed to new and improved methods and systems for breeding swine that makes it economically feasible for many producers that maintain terminal swine to have an SGN at their own facilities under improved direct control by the producers. In another aspect, the invention is directed to methods and systems in which the producer's SGN is genetically linked to a commercial genetics supplier's SGN. In another aspect, the invention is directed to the use of closed SGNs for these purposes thereby additionally providing animal health benefits to the terminal swine producers and ultimately to the meat consumer.

Such new and improved methods and systems for producing and delivering genetic improvement to swine producers consistent with maintaining a high degree of isolation among herds for insuring good animal health are greatly needed and are provided in accordance with the different aspects and embodiments of the invention.

### SUMMARY OF THE INVENTION

The invention comprises the use of two or more genetically linked SGNs for producing and delivering genetic improvement to producers of terminal swine for meat. The SGNs comprise at least a SGN1 (sometimes referred to as SGN1 characterized by a rate of genetic improvement and a rate of inbreeding where the number of animals is sufficient for achieving and maintaining over multiple generations a desired balance between the rate of genetic improvement and the rate of inbreeding and at least one SGN2 (sometimes referred to as SGN2) characterized by a smaller number of animals insufficient to maintain that balance in the absence of periodic introduction of germplasm. Except for animals from the SGN1 that are used to establish the SGN2, the SGN2 is closed to introduction of live animals to greatly reduce or eliminate the risk of health hazards due to introduction of live animals. The SGN is a closed SGN optionally with new germplasm introduced from time to time via semen or embryo transfer (ET) since periodically introducing new germplasm into the SGN1 herd may permit additional genetic improvement. Use of pathogen-free semen for breeding is an advantageous way of introducing new genetics into an SGN without opening the herd. After establishment of the SGN2, germplasm and improved genetics introduction from SGN1 to SGN2 is limited to sperm or optionally embryos produced under conditions insuring freedom from diseases of concern. A key benefit or advantage of closing the SGN2 is to maintain the health of the producer's herds since by closing the herd to live animal introduction, the introduction of unwanted pathogens can be reduced to a significant degree. Data collected from both the SGN1 and the SGN2 are used periodically to provide target measures of genetic improvement and to determine performance measures for the SGN2. Using these methods and systems has been found to enable rates of genetic improvement in the SGN2 to equal or exceed rates of genetic improvement in the SGN1.

According to the invention, the SGN1 and the SGN2 are genetically linked to permit the use of statistical models such as BLUP that can distinguish genetic from environmental effects with phenotypic data collected from both herds including, unless otherwise required by the context, molecular marker data derived from cellular samples from live animals. The genetic linkage is preferably provided by the use of semen from related or identical sires for breeding in both the SGN1 and the SGN2 herds and trait or data linkage is provided by collection and use of at least a core set of phenotypic data from the resulting offspring in both SGNs. Since the SGN2 can be significantly smaller than the SGN1 due to the genetic linkage and trait data linkage, the invention makes possible establishment of an SGN at a swine producer's facility that is under the producer's control to produce improvement in a desired direction without the need for maintaining an SGN having the size and associated costs of the SGN1. Thus, using an SGN2 with a SGN1 as described herein, it is possible to accomplish the producer's goals of substantially or completely eliminating live animal introductions for animal health reasons, gaining control over genetic improvement, and at the same time obtaining the benefits of genetic improvement via the SGN1 that were previously only possible based on maintaining an independent SGN as known in the prior art.

In one embodiment the invention comprises method and system for producing genetic improvement in swine in which a first swine genetic nucleus elite breeding herd or SGN1 is provided or made available at a first site effectively isolated for purposes of preventing transmission of selected pathogens to a second site at which is located a SGN2 derived from the SGN1, the SGN1 having a rate of genetic improvement and a rate of inbreeding and a number of animals sufficient for achieving and maintaining over multiple generations a stable balance between the rate of genetic improvement and the rate of inbreeding, the SGN2 having a smaller number of animals than the SGN1, and the SGN1 and SGN2 further genetically linked by use of semen from the same or related sires in producing offspring in both the SGN1 and the SGN2. Optionally, further genetic linkage can be provided by embryo transfer (ET). This embodiment further includes steps of using a core set of phenotypic data at least some of the traits of which are measured in both the SGN1 and the SGN2 herds and generating a ranking of dams in the SGN2 for achieving a targeted measure of genetic improvement for a next succeeding generation in the SGN2 and using semen provided from sires in the SGN1 for use in breeding dams in the SGN2 to achieve the targeted measure of genetic improvement in the SGN2. According to a further aspect, the measure of actual genetic improvement is also periodically determined and provided to the producer of the SGN2.

In another embodiment, the invention comprises method and system for producing genetic improvement in swine comprising, relative to a first swine genetic nucleus elite breeding herd or SGN located at a first site effectively isolated for purposes of preventing transmission of selected pathogens, maintaining a second site at which is located a SGN2 derived from the SGN1, the SGN1 having a rate of genetic improvement and a rate of inbreeding and a number of animals sufficient for achieving and maintaining over multiple generations a stable balance between the rate of genetic improvement and the rate of inbreeding, the SGN2 having a smaller number of animals than the SGN1, and the SGN1 and SGN2 being further genetically linked by use of semen from the same sires in producing offspring in both the SGN1 and SGN2. This embodiment further includes steps of selecting dams for breeding from a ranking of dams in the SGN2 for achieving a targeted measure of genetic improvement for a next succeeding generation in the SGN2, and breeding the selected dams using semen from sires in the SGN1 selected for use in breeding dams in the SGN2 and periodically providing determined measures of actual genetic improvement to the producer of the SGN2 to achieve the targeted measure of genetic improvement in the SGN2.

According to another aspect, the invention comprises method and system for determining measures useful in breeding swine comprising accessing at least a core set of phenotypic data obtained from each of a first swine genetic nucleus breeding herd SGN1 and a second swine genetic nucleus herd SGN2, the SGN1 and the SGN2 being genetically linked; and producing measures for at least one of the SGN1 and the SGN2 herds selected from the group consisting of measures of estimated breeding values for selected traits and measures of rate of genetic improvement and combinations thereof.

In accordance with other aspects of the invention, the invention comprises methods and means for assisting the terminal swine producer in managing the breeding and cross-breeding of animals derived from an SGN1 herd to improve genetic potential in an entire swine production system involving multiple generations derived from the producer's own SGN2 herd, for example, GGP (great grandparent), GP (grandparent), PS (parent swine) and multiple line crosses for producing MS (market swine or terminal swine “TS”) used only for meat and by-products. In other aspects, the systems and methods in accordance with the invention can be more cost effective and profitable for the swine producer than prior art systems even without placing a value on health benefits.

### BRIEF DESCRIPTION OF THE DRAWINGS

FIG. 1 illustrates schematically swine production systems in accordance with the prior art and a swine production system in accordance with the invention comprising use of genetically linked SGN1 and SGN2 and optionally other SGN herds.

FIG. 2 illustrates schematically establishment and maintenance of SGN2 maternal line herd that corresponds to and is genetically linked to a SGN1 maternal line herd.

FIG. 3 illustrates schematically an embodiment of information flows used in accordance with an aspect of the invention.

### DETAILED DESCRIPTION OF THE INVENTION

The invention is directed to improvements in the breeding and production of animals to produce market swine. The swine lines to be bred can be selected from any breed of swine. Breeds or lines of swine, as those terms are generally used today, are animals having a common origin and similar identifying characteristics. Lines of swine are groups of related animals produced, for example, but not exclusively, by line breeding, the mating of animals within a particular line according to a mating system designed to maintain a substantial degree of relationship to a highly regarded ancestor or group of ancestors without causing unacceptably high levels of inbreeding. In particular aspects, the invention relates to improvements in the breeding of maternal lines for the production of market pigs, though the invention can be used with paternal lines and other lines as well. A maternal line, as is well known, is a line that excels in the maternal traits of fertility, freedom from dystocia, milk production, maintenance efficiency, and mothering ability; while paternal lines are strong in paternal traits such as rate and efficiency of gain, meat quality, and carcass yield.

The invention comprises methods and systems for producing genetic improvement in swine in which a SGN1 (first SGN—“swine genetic nucleus elite breeding herd”) at a first site and a second SGN (“SGN2”) at a second site closed to live animal and associated pathogen introductions are used cooperatively to effect genetic improvement in SGN2. According to an aspect of the invention, a measure of genetic improvement is selected and a target measure of genetic improvement is set for SGN2 in a future period and a measure of achievement of genetic improvement (“performance measure”) is determined for SGN2 at intervals during and following the period and is provided to the SGN2 producer. As illustrated, the preferred measure of genetic improvement is the ratio i/t usually referred to by geneticists as the rate of genetic improvement. The term i is described in more detail below, but may be referred to as the selection intensity for a selected criterion expressed in standard deviations. For swine, the generation interval t is usually defined as the average age of the parents at the time of farrowing of offspring for the next generation. The target measure and the performance measure can be periodically provided directly to the SGN2 producer or can be compared to the target measure or to performance measures of other SGN herds to evaluate successful implementation of genetic improvement or to determine the existence of and be used in assessing correction of problems in SGN2 or to establish the target measure for a succeeding interval.

The ratio i/t for swine can vary over positive and negative numbers around zero up to an upper value that may approach a biological limit for a given population. For dams, a reasonable upper value is about 1.50 (assuming use of gilts for breeding to minimize generation interval t) while for sires a reasonable upper value is about 2.0 although in both dams and sires somewhat higher values can also be observed with the implementation of improved reproduction technologies. Sire upper limits tend higher than dam upper limits since intensity i for sires can be higher than for dams, which require a higher number of replacements, and therefore cannot as a practical matter be subjected to the same selection intensity as the sires. Use of embryo transfer (ET) can further increase the i/t ratio for dams in the direction of that attainable for sires. Since it has surprisingly been found that in the absence of target and performance measures, notwithstanding all of the other components of the described breeding systems, the performance of the SGN2 herd typically is on the order of about 0.3 or even less to less than about 0.5, a useful practical measure of achievement in SGN2 is greater than about 0.5, more preferably greater than about 0.7 or 0.9. Most preferably rates of improvement of 1.1, 1.3 or even 1.5 can be achieved.

As indicated, the preferred measure of genetic improvement is the rate of genetic improvement or i/t where i is selection intensity expressed as the difference between the mean selection criterion of those individuals selected to be parents and the average selection criterion of all potential parents expressed in standard deviation units and t is the generation interval measured in years. In this instance, the target measure will be the predicted annual rate of genetic improvement of SGN2 in standard deviation units and the performance measure will be the actual rate of improvement of SGN2 again in standard deviation units. As is known, determination of i/t for SGN2 requires knowledge of i for both dams and sires and therefore requires a collection and an exchange of relevant information (e.g., i/t for sires for SGN1 and i/t for dams for SGN2) to permit determination. Accordingly, a further aspect of the invention comprises methods and systems to provide the relevant information to enable determination of i/t for SGN2 to and for generating the target measure and determining the performance measure and for generating further target measures.

Both target and performance measures are highly desirable and advantageous for achieving the desired results of systems where SGN1 and SGN2 are used to produce genetic improvement in SGN2. In fact, in the absence of such measures the rate of genetic improvements in herds has been found to be on the order of 0.3 to less than about 0.5 although theoretical rates of improvement were much higher. It appears that periodically providing the performance measures insures conducting the other operations to achieve the targeted results, as well as, more concretely, leading to rapid identification and correction of operating problems.

According to various embodiments, the invention comprises methods and systems for producing genetic improvement in swine in which an SGN1 herd is provided or made available at a first site effectively isolated for purposes of preventing transmission of selected pathogens to a second site at which is located an SGN2 herd. As used herein, a first site will be effectively isolated from the second site if the SGN2 is totally isolated from other swine, for example, not within a radius of a minimum of about 3, to about 5 miles or even more (10 miles or more) from another herd, and if there are strict biosecurity procedures followed at the SGN2 site controlling human, animal and vehicular traffic, and if (preferably) the initial stocking of SGN2 from SGN1 occurs all at one time. If additional stocking after initial stocking is to be used, the subsequent stocking must flow through a quarantine facility to properly screen for pathogens.

According to various embodiments, the SGN1 herd has a rate of genetic improvement and a rate of inbreeding and a number of animals sufficient for achieving and maintaining over multiple generations a stable balance between the rate of genetic improvement and the rate of inbreeding in the SGN1 herd.

To illustrate these aspects for SGN1, it is useful to look at the key equation for genetic change in a herd. The key equation states that the rate of genetic change in a selection criterion for any given trait (for example, estimated breeding values—“EBV”, or other phenotypic information used as a basis for selection for that trait) is directly proportional to three factors: accuracy of selection, selection intensity, and genetic variation; and inversely proportional to a fourth factor: generation interval. Mathematically, for any given trait, the key equation can be written
Δ_{BV}/τ=r_{BV,B\hat{ }V}σ_{BV}i/t, (1)
where Δ_{BV}/τ is the rate of genetic change per unit of time τ, r_{BV,B\hat{ }V} is the accuracy of selection (correlation between estimated breeding values and true breeding values for a trait under selection), σ_{BV} is the genetic variation for the trait of interest, i is selection intensity expressed as the difference between the mean selection criterion of those individuals selected to be parents and the average selection criterion of all potential parents expressed in standard deviation units, and t is the generation interval (average of parents' ages at the time of farrowing). (It is noted in passing that this equation also provides guidance for determining i/t since the rate of genetic improvement of any given trait (i/t) can be isolated from the equation.) This equation is well known and capable of readily being used by those skilled in the art. From the equation, it will be clear that the producer's impact on the rate of genetic change will be primarily controlled by increasing selection intensity and by decreasing generation interval t to the extent practical.

In commercial breeding operations, a goal is to achieve an advantageous rate of genetic improvement, avoid disadvantageous levels of inbreeding, and maintain herd costs at an economically advantageous level. This creates a tension between having a few highly elite animals for breeding to increase selection intensity and reduce breeding costs and having a large number of breeding animals to prevent inbreeding depression. Consequently, it is advantageous to design breeding programs so as to balance the Rate of Response and the Rate of Inbreeding and if possible find an advantageous balance between the two consistent with economy of operation. This process in general terms is well known in the art and need not be further explained here, except for convenience to note that the rate of response per generation is illustrated above by equation (1) and that the rate of inbreeding and its relationship to breeding herd size can be illustrated by equations (2) and (3) below:
ΔF=½Nₑ (2)
where ΔF is the rate of inbreeding per generation and Nₑ is the effective population size of the breeding population:
Nₑ=4NₘN_{f}/(Nₘ+N_{f}) (3)
where Nₘ and N_{f} are the number of males and the number of females used as parents for each generation. By iterative use of these formulas or computer modeling of these formulas for desired traits and rates of response and availability of facilities for stocking and breeding, persons skilled in the art readily determine advantageous herd sizes for each specific situation encountered in breeding and can likewise determine appropriate herd sizes in accordance with the invention in light of the disclosure herein.

According to embodiments of the invention, the methods and systems according to the invention can be used for any nucleus herds used in breeding. It has been found particularly advantageous to use the invented methods and systems in connection with breeding and production of maternal line parent dams for breeding by paternal line terminal sires for the production of terminal swine because of the significantly larger number of dams otherwise required and the corresponding greater benefit to be obtained from the methods and systems disclosed herein. As a result, a preferred embodiment described herein relates to breeding and production of maternal line parent dams, but the invention can be readily applied by those skilled in the art to other nucleus breeding systems and for other lines including paternal lines.

According to an aspect of the invention, for example, referring to nucleus maternal line herds, both the genetics supplier's maternal line SGN herd (sometimes referred to as SGN1) and the producer's maternal line SGN herd (sometimes referred to as SGN2) can be smaller than otherwise would be necessary to achieve advantageous results. The two herds (SGN1 and SGN2) must be genetically linked as discussed in more detail below. The extent to which the genetics supplier's herd can be reduced in size depends in part upon whether the phenotypic data collected by the producer is sufficiently accurate and reliable to meet the supplier's requirements for data to be used in determining EBVs for the genetics supplier's herds. In any event, it will be immediately clear to the skilled person that the SGN2 herd can be much smaller when it is genetically linked to and supported by accurate information from the SGN1 herd than would otherwise be possible.

Referring in more detail to an exemplified nucleus maternal line herd, as a practical matter, it will usually be desirable to have no fewer than about 50 dams in the SGN2 herd to provide at least about 3 dams coming into heat on a weekly basis to provide an advantageous rate of breeding work for planning and staffing purposes. As described below, in a preferred embodiment, no sire or boar stud herd is maintained for the second nucleus herd because semen from the SGN1 herd is obtained and used to provide genetic linkage between the herds. The upper boundary of herd size for the producer's SGN2 herd will be determined by the breeding program and number of parent sows required for producing the desired number of terminal swine on a regular basis as well as producing replacement swine. Thus the breeding SGN2 herd can range from about 50, which can support up to about 50,000 parent dams per year, to about 100, which can support about 100,000 parent dams per year, or to about 1000, which can support up to about 1,000,000 parent dams per year, or can take other values depending on the number of parent dams to be produced each year.

Referring now to the first nucleus maternal line herd SGN1 to illustrate the effects of aspects of the invention on herd size in the genetics supplier's maternal line herd, consider, for example, that a minimum size to prevent unacceptable inbreeding in the SGN1 herd has been found to be about 450 sows. Additionally, of course, it is necessary to increase the dam herd by a number sufficient to provide replacement dams for the dam herd and boars for the stud herd. Further increases in size will be necessary if culls (animals excluded from selection for breeding) constitute a significant portion of the offspring or for other reasons such as health or to provide a sufficient population for a desired weekly breeding schedule or the like. As a result of using the invention, and even without use of the external herd SGN2 data for calculating EBVs for internal (to the genetics supplier) SGN1 breeding purposes, it will be possible to reduce the size of the SGN1 herd toward the minimum size needed to balance rate of response and rate of inbreeding by significantly reducing the number of animals maintained for replacement breeding due to that portion of the herd necessary for production of those dams having been in effect transferred to the SGN2 herd.

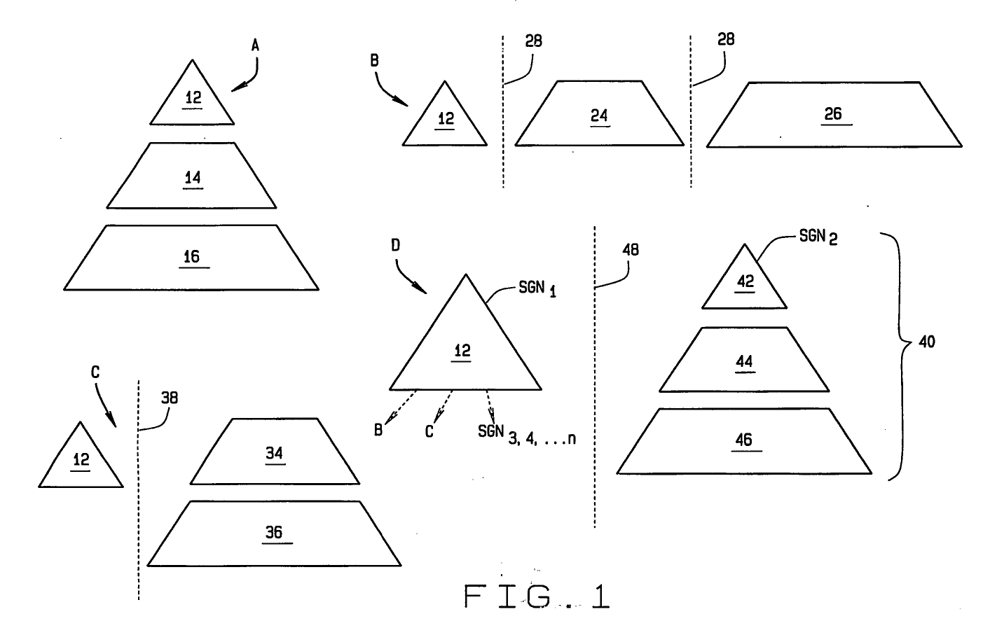

Further advantage can be obtained if the producer's collected phenotype data can be used in determining EBVs for the genetics suppliers SGN1 herd. For example, when a producer initiates an external genetic nuclear herd system as described herein, there may be a delay between when the producer's data collection practices are sufficient to result in reliable improvement in SGN1′ herd and the time when the data accuracy meets the genetics supplier's standards. However, as soon as the data can be reliably used for determining EBVs for the genetics supplier's SGN1 herd, it is apparent that the genetic supplier will be able to increase accuracy of prediction for selected traits. Likewise, referring again to FIG. 1, as additional external closed SGN herds (SGN3, SGN4, etc.) come into use that are also genetically linked and data linked to SGN1, further improvements in accuracy can be achieved, provided that a sufficient size herd to prevent unacceptable inbreeding is maintained.

According to various embodiments, as can be seen from the above discussion, the SGN2 can have a significantly smaller number of animals than the SGN1. The size for the SGN2 herd is targeted at a minimum of 50 sows or the appropriate number of female animals to provide breeding dams for cross-breeding as practiced for producing parent dams used for producing terminal swine, and additionally replacement dams for the SGN2 herd itself. Another consideration influencing SGN2 size includes providing enough dams to provide a steady average supply of replacement females on a regular (e.g., weekly) basis to permit the effective use of facilities, labor and supplies.

According to the invention, the SGN1 and SGN2 are genetically linked by use of semen from the same or related sires from SGN1 or by use of embryo transfer or other advanced reproduction techniques to produce offspring in both the SGN1 and the SGN2 herds that to some known degree share a specifiable pedigree. According to a preferred aspect of the invention, the desired genetic linkage is provided by using sires from the SGN1 to provide semen for breeding both of the SGN1 and SGN2 herds. The result is that, all of the offspring of the prescribed matings in both herds are half-sibs at least in respect of certain animals in each herd, i.e., there are groups of half-sibs sharing a common pedigree, and phenotypic data collected from both herds are capable of being jointly processed using conventional and available BLUP computer programs. Other means of providing genetic linkage can also be utilized, including but not limited to use of semen from related sires and other techniques useful for causing offspring to share a pedigree as herein defined.

According to further aspects of some embodiments of the invention, the invention includes steps of generating a ranking of dams in the SGN2 for achieving a targeted measure of genetic improvement for a next succeeding generation in the SGN2 and using semen provided from sires in the SGN1 for use in breeding selected dams in the SGN2 to achieve the targeted measure of genetic improvement in the SGN2. Likewise, according to some aspects of the invention, performance measures are likewise determined using information from both SGN1 and SGN2 and periodically provided to the SGN2 producer.

A key priority in producing reliable phenotypic data is the clear identification of the measurements (“tests”) to which the swine will be subjected and the conditions under which the swine will be maintained during the test period. For maternal line swine, the test period typically begins upon farrowing and ends upon the making of a decision for which testing was prerequisite, such as return of offspring to the parent herd as a replacement animal, shipment to market, and the like, after which the offspring may be said to be “off-test”. As discussed below in more detail, a minimum set of data comprises reproduction data, a more extensive set of data comprises reproduction data and growth rate data (e.g., weight at off-test and the like), and a very advantageous set of data comprises reproduction data, growth rate data and predicted carcass data. Many measures of those traits and techniques for making measurements of those traits are known to those skilled in the art, and while a few of these are specifically described herein, the invention is not limited to those mentioned but extends to all phenotypes and tests useful for breeding purposes in accordance with the different aspects of the invention.

To illustrate the generation of ranking of dams in the SGN2, consider first that the phenotypic measurements required will depend on the traits being enhanced. For maternal lines, desirable traits and data include for purposes of illustration at least reproduction traits and data such as litter size, underline, and the like, growth traits and data such as growth rate such as weight at off-test, and carcass traits and data such as percent lean, back-fat and loin-eye area measurements. The SGN1 herd can be preferably evaluated for all of the traits mentioned above and the SGN2 herd is preferably evaluated for at least reproductive traits and more preferably for one or more of the growth traits and carcass traits.

To increase the accuracy of EBV predictions it is of course highly desirable that the measurements of both herds are made using the same techniques and most preferably using personnel who have received substantially the same quality training in using those techniques.

According to aspects of the invention, the resulting phenotypic data can be used to produce rankings, for example of dams in the SGN2 herd, from which the most desirable animals for achieving the targeted improvement can be bred with semen from SGN1. Preferably, the rankings are generated using BLUP computer programs to which data from both the SGN1 and the SGN2 herds can be input.

As indicated above, it may be desirable in some instances, but not in others, to use data from the SGN2 herd for generating EBVs for the SGN1 herd. Flexibility in this respect can be achieved by giving data from each animal indexes indicative of source and location and by making modifications either to the input routines or to the BLUP programs themselves to access the appropriate data for the herd and traits whose EBVs are being determined. Likewise, the outputs from BLUP programs, if in the values of the traits measured, can be readily weighted using economic information to maximize economic value for each assortment or constellation of traits. Such input and output routines and modifications are well known to those skilled in the art and can be readily implemented if not already available in the BLUP programs being used.

As previously discussed, the BLUP programs are well known and commonly used in swine breeding and are readily available to those skilled in the art. An advantageous system for producing BLUP values is the MTDFREML system available from Dale Van Vleck at the University of Nebraska—Lincoln. Other systems known and available to those skilled in the art can also be used and adapted for use with data as described herein by the routine exercise of programming skills.

In most SGNs, although it is possible to synchronize estrus by weaning, as a practical matter females are bred as they come into estrus during the course of the year. In contrast, elite sires or semen from elite sires may be available at most times unless the demand for semen is excessive. Consequently, according to an aspect of the invention, it is useful to generate the dam rankings on a periodic, preferably weekly, basis using currently available data so that as individual elite dams come into estrus they can be bred using semen from available elite boars. Desirably, the herd size is managed so that on average a certain number, for example, 3 or more come into estrus each week, since a predictable rate of females in estrus permits determining the amount of semen or size of the boar stud herds that will be required as well as spreading personnel and facilities costs throughout fiscal periods. According to a preferred aspect of the invention, the dam rankings for the SGN2 herd can be generated at any convenient period or interval, e.g., monthly or preferably weekly to provide very advantageous results in implementing a breeding program to improve herd genetics. Generally, most large producers practice weekly flow and weekly reports are most advantageous. Accurate information reporting from the producer herd site to the genetics supplier site is required, reporting bred and farrowed dams, so that the weekly dam rankings consists only of “open” dams, that is, dams that are not bred and are available for breeding.

Since the dam rankings are provided to the SGN2 producer, that producer has an increased measure of control of improvement in the SGN2 herd compared to prior art use of only an SGN1 herd. For example, by maintaining an SGN herd and increasing selection pressure, the SGN2 herd can potentially achieve progress at a faster or slower rate in respect of selected characteristics relative to the SGN1 herd. A major factor in achieving desirable rates of genetic improvement as indicated by key equation (1) above is provided by generation interval and to the extent that the producer breeds lower parity females and highest ranked females, the producer further can further accelerate genetic improvement. Also, since the data for the SGN2 herd can be collected by the SGN producer, additional effort to achieve good data structure and accuracy will also lead directly to improved genetics in the SGN2 herd. All of these advantages can be achieved in accordance with the invention while maintaining the advantages of having SGN1 and SGN2 herds closed relative to each other following the initial establishment of the SGN2 herd.

According to an aspect, the invention includes a feature of using the steps of generating a ranking of dams in SGN2 and using semen provided from sires in SGN1 for use in breeding dams in the SGN2 to achieving a targeted measure of genetic improvement for a next succeeding generation of SGN2. To illustrate this point, consider that the two herds SGN1 and SGN2 could be established and remain closed to each other after initial establishment, thereby achieving the animal health benefits of the present invention, and semen from selected sires in the SGN1 herd could be used to transfer genetic improvement to SGN2. However, in the light of the present invention, it can be seen that this system would not be capable of producing a targeted measure of improvement in the SGN2 herd because of the absence of dam selection in the SGN2 herd. In retrospect also, it can be seen that in order to accomplish dam selection, it is necessary to collect data representative of some of reproductive, growth and carcass traits and more importantly to use that data to generate the ranking of dams. Even more important was the recognition that the use of semen from SGN1 to close SGN2 for health reasons also provided a genetic linkage between the two herds that permitted determination of EBVs for SGN2 in addition to SGN1.

According to another embodiment of the invention, the invention comprises method and system for breeding swine comprising determining measures for breeding swine. At least a core set of phenotypic data obtained from each of a first swine genetic nucleus breeding herd SGN1 and a second swine genetic nucleus herd SGN2, the SGN1 and the SGN2 being genetically linked is accessed; and measures for at least one of the SGN1 and the SGN2 herds selected from the group consisting of measures of estimated breeding values for selected traits and measures of rate of genetic improvement and combinations thereof are produced. In a further aspect, measures are produced for each of the SGN1 and SGN2 herds. In yet further aspects, at least a core set of phenotypic data from each of an additional set of swine genetic nucleus breeding herds is accessed, each SGN genetically linked with at least one other of a resulting total set of swine genetic nucleus breeding herds SGNs; and measures are further produced for at least one of the resulting total set of SGNs.

According to preferred aspects of this embodiment of the invention, the measures of estimated breeding values or of genetic improvement are determined using a best linear unbiased prediction (BLUP) statistical model. For example, phenotypic data relevant to selected traits from at least one of SGN1 and SGN2 can be provided by a data link to a database that is data linked to a data processor for producing the measures of estimated breeding values, and then the data processor is used to access the database to produce the measures of estimated breeding values or of rate of genetic improvement.

According to more preferred aspects of this embodiment of the invention, the measure is a measure of rate of genetic improvement for at least one of SGN1 and SGN2 and the invention comprises producing a measure of rate of genetic improvement for at least one of SGN1 and SGN2; and the measure of genetic improvement is provided by a data link to a site associated with the swine genetics breeding herd for which the measure is produced. At the site where the SGN is located, the measures are then used, for example, for measuring compliance with a predetermined breeding plan for the SGN associated with that site or for improving compliance with the breeding plan upon occurrence of a provided rate of genetic improvement differing from a target rate of genetic improvement associated with the breeding plan or for adjusting a target rate of genetic improvement associated with the breeding plan upon occurrence of a provided rate of genetic improvement differing from the target rate.

According to another aspect of the invention, a ranking of dams in the SGN2 herd is periodically generated for achieving a targeted measure of genetic improvement for a next succeeding generation in the SGN2 herd; and the ranking is provided for use for selection of dams for breeding using semen from SGN1 selected for use in breeding dams in the SGN2 to achieve the targeted measure of genetic improvement in the SGN. For highly advantageous results, this ranking of dams is generated and provided weekly to the SGN2 producer.

Commercial breeders usually desire to take advantage of line or breed complementarity, an improvement in the overall performance of crossbred offspring resulting from crossing lines or breeds of different but complementary biological types. In swine, line complementarity typically comes from crossing maternal lines (lines that excel in maternal traits such as fertility, litter size, mothering ability, and maintenance efficiency) with paternal lines (lines that are strong in paternal traits such as rate and efficiency of gain, meat quality and carcass yield) where the maternal and paternal lines are complementary to each other. The ultimate in line complementarity is achieved in terminal-sire crossbreeding systems in which maternal-line females are mated to paternal breed sires to produce progeny that are especially desirable from a market standpoint. Daughters of terminal sires are not kept as replacements but are sold along with their male counterparts as market animals.

According to the invention, there are provided methods and systems for breeding and producing terminal swine for meat. The invention is illustrated using a crossbreeding swine production system utilizing both maternal lines and paternal lines for ultimately producing animals for meat, and the closed external SGN herd described herein specifically relates to a closed external SGN herd for producing maternal line dams that can be bred with paternal line terminal boars for producing terminal swine for meat production. However, the invention is not limited to the particular embodiment described but can be extended to any system for genetic improvement of swine lines in which (1) a central SGN herd and at least one external SGN herd (2) are bred using at least one parent, usually the sire, of known genotype to provide genetic linkage between offspring of the two herds sufficient for jointly processing data from both herds using currently available best linear unbiased prediction (BLUP) programs, (3) relevant data for determination of EBVs of potential parents in at least the external SGN herd are collected from offspring of both herds; (4) EBVs are determined for at least potential dams in the external SGN herd and (5) the external SGN herd is genetically improved by selection using the resulting EBVs calculated from both herds with imposition of target rate of genetic improvement criteria such as i/t for the SGN2 herd.

Referring now to FIG. 1, FIG. 1 illustrates schematically swine production systems in accordance with the prior art at A, B, C and a swine production system in accordance with the invention at D comprising use of both a central nucleus breeding herd SGN1 and an external nucleus breeding herd SGN2. In D, the maternal line stud and dam herds are preferably isolated and all breeding is controlled in accordance with a breeding program determined as described herein. Thus, at A, there is illustrated the prior art use of a central SGN1 herd at 12 that is used to provide genetically-improved maternal line boars (semen) or sows or both to a multiplier facility 14 that in turn is used to provide dam lines for producing terminal pigs for feeding, finishing and harvesting 16. While system A illustrates all functions without segregation of entity or space among the various functions, in practice the functions are usually separated among entities or space or both as schematically illustrated at prior art systems B and C in which dashed lines 28 and 38 indicate different entities or different locations or both, and in which the reference numerals of B and C (and D illustrating the invention discussed below) correspond to those of A by their final digit.

Referring now to system D, D illustrates a system in accordance with the invention in which a swine genetics provider 12 having a SGN1 herd, in addition optionally to supplying swine genetics to traditional facilities B or C or both, also on a one-time or infrequent basis provides stock to establish an external SGN maternal line dam herd (or a plurality of mate mal line dam herds) at a producer facility D. (In addition to the single external SGN herd shown at D, there may be one or more other external SGN herds SGN_{1,2, . . . n} also established for other producers or facilities and these additional herds can benefit both genetics supplier 12 and producer D as explained in more detail herein.

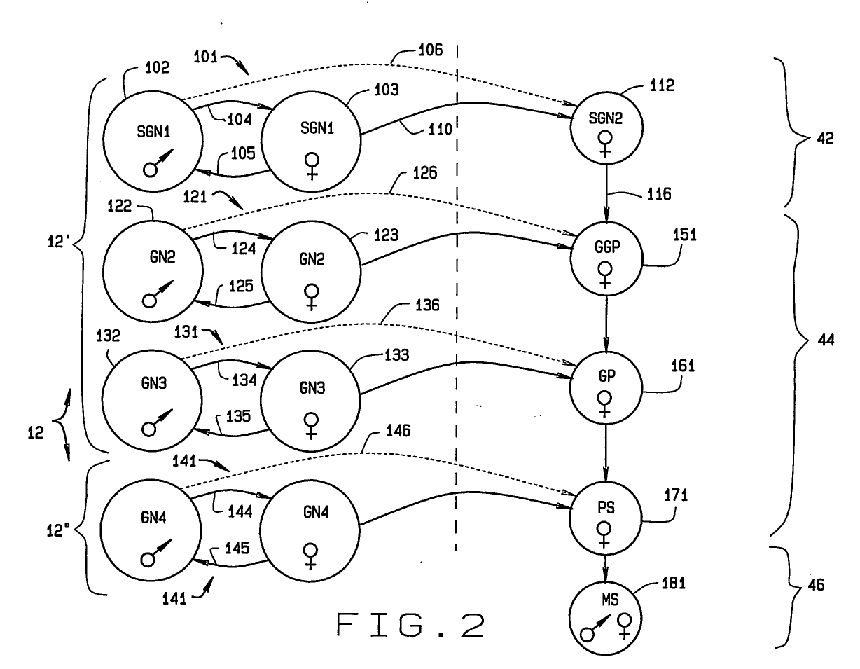

Referring now to FIG. 2 in detail, FIG. 2 illustrates D in FIG. 1 in greater detail. Specifically, FIG. 2 illustrates that the SGN herds 12 of the genetics supplier of FIG. 1 may comprise a plurality of pure line dam herds 12′ and pure line sire herds 12″ which can be used for producing market swine (MS). As illustrated by line 110, at the time of initial establishment of the external SGN2 maternal pure-line herd 42, live female animals of SGN1 stock, optionally bred sows, may be provided after thorough health screening on a one-time or at least non-routine basis to the producer as shown by herd 112. At the time of establishing herd 112, maternal line semen from stud herd 102 can also be provided (illustrated by dashed line 106 from the genetics provider for initially breeding the SGN2 females by single sire matings resulting in offspring. Thereafter, the producer selects elite dams from the SGN2 offspring for breeding with elite boars whose semen is provided by line 106 from the genetics supplier. With good testing, selection and mating practices, it will therefore be possible to impose selection intensity to improve the SGN2 herd in some instances at a more rapid rate than is necessarily accomplished in the SGN1 herd.

Those skilled in the art will appreciate that the result of using semen from the same sires for breeding SGN1 herd 103 and for breeding SGN2 herd 112 is that the prescribed offspring from both herds will be half-sibs, that is, there are groups of half-sibs in both herds, sharing a common pedigree and therefore that EBVs can be determined for both SGN1 herd 103 and SGN2 herd 112 using conventionally available BLUP programs. To illustrate, by using single-sire matings for all females, it will be known that the male selected for each female sired all offspring from that female of SGN2 herd 112. Assuming for purposes of illustration that the goal of breeding is to enhance production of large numbers of dams with large litter sizes, good mothering ability, good growth rate and good carcass characteristics, the offspring at birth can be tagged with a unique animal identification. All females from litters in SGN2 showing specific abnormalities such as atresia ani, scrotal rapture, cryptorchidism or hermaphroditism are not eligible for the SGN breeding pool and can be culled. The remaining females may be taken off-test at the same time (about 165 days), weighed, and tested for backfat and loineye area, and thereafter closely evaluated for physical characteristics per selection guidelines until final selection for being returned to SGN2 herd 112 for breeding. The resulting collected data of the non-culled animals can then be processed by BLUP to provide EBV ratings for each animal and the EBV ratings can be provided to the producer for use in selecting females in herd 112 for breeding.

Concurrently, the sires of herd 102 can be culled, tested, and selected in a similar way and EBVs determined for potential sires for the next breeding of females in herd 112.

According to aspects of the invention, the producer of herd 112 can establish a targeted measure of genetic improvement or change. In general terms, about half of the improvement will derive from the sires selected for breeding from herd 102 and about half of the improvement will derive from the dams selected for breeding from herd 112. As a practical matter, since a single boar can be used to breed a number of sows, much of the selection pressure on herd 112 will derive from scrupulously following selection guidelines within herd 112. The regular reporting of actual genetic improvement has proved to be instrumental in achieving results that theoretically could have been achieved without the weekly reporting of actual improvement measures. The feedback loop created by providing the results actually obtained facilitates fine tuning of the practices of herd 112 management and actually permits the targeted measures of improvement to be achieved.

In this system, it will be seen and appreciated by persons skilled in the art that by using semen from genetics provider 12 in external SGN herd 42, the resulting animals are preferably all half-sib animals of corresponding animals in the genetics provider's central SGN herd 12 that may be produced using the same maternal line boars. Use of related rather than identical sires results in a lower, but still useful for BLUP, degree of genetic relationship. This use of common male genetics permits phenotypic data collected from animals produced in external SGN herd 42 and phenotypic data collected from half-sib animals in the originating SGN herd 12 to be processed using BLUP to produce EBVs without the necessity of maintaining the external SGN herd at a number of animals which would by itself be sufficient to guarantee a sufficient level of heterozygosity and control of inbreeding in the external SGN herd. Simultaneously, it permits EBVs determined for the central SGN herd to have a greater accuracy level than would have been possible in the absence of the external SGN herd. As more external SGN herds are established as illustrated by D′ in FIG. 1, further efficiencies can be achieved.

To illustrate, if additional external herds SGN3, SGN4, etc. are added, the resulting increased numbers of related animals permits more precise estimation of EBVs for all of the herds whose data can be used. Moreover, comparison of target measures and performance measures of genetic improvement over a number of sites helps to identify facilities that are failing for one reason or another to achieve the rates of improvement that they are capable of achieving and therefore provides a measure of competitive efficiency for the operators of those facilities.

Referring again to FIG. 2 and in particular to the multiplier function 44, during multiplier function 44, producer 40 can further breed selected maternal line females derived from the SGN2 herd in accordance with the invention using semen or boars derived from genetics supplier 12 as indicated by dashed line 126 to produce great-grandparent stock (GGP) as illustrated by 151. As, illustrated, the multiplier function 44 can include steps cross breeding using a number of pure lines from the genetics supplier herd GN2, GN3, and the like, for example, as illustrated by lines 121 and 131, consisting of sire herds 122 and 132 and dam herds 123 and 133 respectively to produce GGP and GP (grandparent) dam herds 151 and 161 respectively. As illustrated, producer 40 can obtain semen for each of the intermediate breeding steps from the genetics supplier via lines 121 and 131. The end result of these breeding steps is the production of a sufficient number of parent swine (PS) dams 171 for breeding with a external terminal boar line illustrated by GN4 whose semen can be provided for example as illustrated by line 141 to produce market swine (MS).

According to a preferred embodiment of the invention, as illustrated in FIG. 2, the only introduction of live animals illustrated by solid line 110 from the genetics supplier to the producer occurs on a one-time basis at the time of establishing the external SGN2 herd. All other genetics introduced into the producer's facilities is via semen as indicated by dashed lines 126, 136 and 146. In the instance of the SGN2 herd 112 and optionally derived dam herds 151, 161, and 171 it can be desirable for the producer to establish boar stud herds for use with the producer's dam herds. By limiting live animal introductions in this way, the external SGN2 herd is a “closed” herd, that is, not open to further live animal introductions, and as a result will advantageously isolate the herd from negative health impacts that result from live animal introductions.

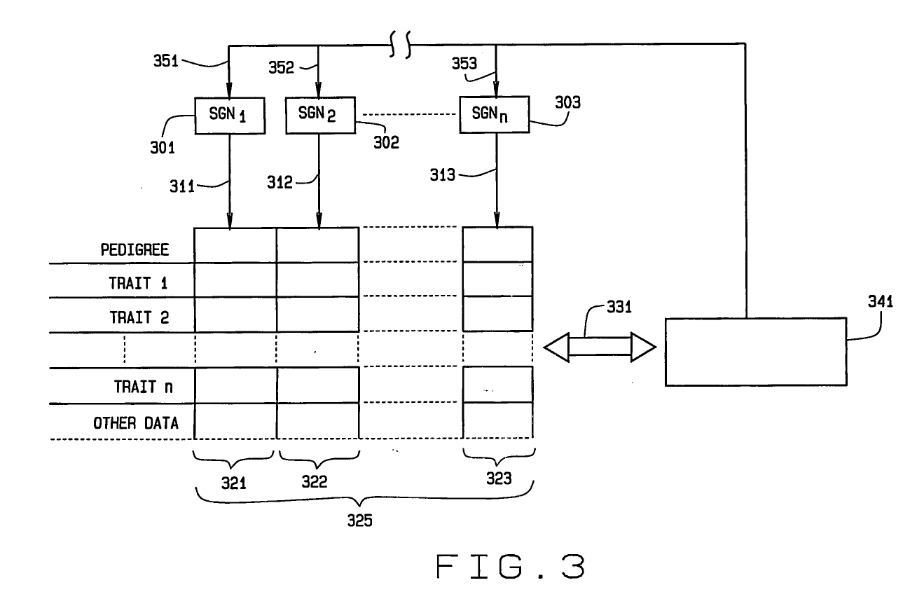

Following establishment of the external SGN2 herd, genetics provider 10 and producer 40 implement data collection and analysis suitable for determining EBVs of the external SGN2 herd using BLUP. While there are many ways this can be accomplished, FIG. 3 illustrates the core of the process. Referring now to FIG. 3 in detail, FIG. 3 illustrates a system for providing, accessing and processing phenotypic data collected from the SGN1, SGN2, etc. herds as described herein and processing the data to provide EBV data and dam rankings to the producer 40 and measures of target and performance measures of genetic improvement as described herein.

As illustrated in FIG. 3, animal identifier and phenotypic data collected from each of SGN1, SGN2, and SGNn herds referenced by 301, 302, and 303 are provided by data links 311, 312, 313 respectively to a plurality of databases 321, 322, 323, or optionally all databases can be part of a single database as illustrated by reference numeral 325. As used in this application, the term data link is used to refer to all input, output, and transmission devices and methods that can be used to provide data in electronic form from various sites to the data base and to return data to the appropriate sites. Thus, the term can include hand-helds, laptops, personal computers, scanners, and the like as input devices, electronic links both wired and wireless, via the internet or via other data connections from input/output devices to the data base and to the sites where information is used. Such matters are well known in the art and those skilled in the art can develop many systems in accordance with the teaching of the invention to accomplish the data transfer, processing and use.

The animal and phenotypic data provided to the databases can be animal identifier data, pedigree data, and phenotypic measurements for Traits 1, 2, . . . , n as illustrated as well as other useful data identified for particular applications as is known to those skilled in the breeding arts. Since, as illustrated in FIG. 2, semen for breeding SGN1, SGN2, . . . , SGNn comes from the SGN1 herd, it will be appreciated that sire phenotypic data for all the herds will be input from the SGN1 site in the usual instance while the dam phenotypic data will be input from the respective herds containing the dams.

Periodically, a data processor 341 accesses selected data from database 325 or its constituent data sets 321, 322, . . . , 323 and obtains the relevant phenotypic data representative of SGN1, SGN2, . . . , SGNn herds for determining EBVs and economically-weighted EBVs of the dams of selected herds using BLUP and to provide a ranking of dams in each herd for selecting dams for breeding. Likewise, the data processor accesses selected data from the database and selects sires most suitable for breeding to achieve desired characteristics in the offspring. The ranking data is provided, for example, by data links 351, 352, . . . , 353 back to the respective herds where the data is needed for implementing a breeding program, including selection of sires and ranking of dams for semen provision and breeding respectively.

According to another aspect, the animal and phenotypic data for each SGN are used to generate measures of genetic improvement for each SGN and to provide those measures back to at least the respective SGN. As illustrated by the double-headed arrow 331, the measures of genetic improvement can also be stored in the database 325. Preferably, both the dam rankings and the measures of genetic improvement are determined on a regular basis, for most advantageous results to accomplish a targeted measure of genetic improvement on a weekly basis, though other intervals can also be useful.

While the invention has been illustrated in terms of various embodiments embodying various aspects, the invention is not limited to the embodiments described herein in detail but by the claims appended hereto interpreted in accordance with applicable principles of law.

1. A method for producing genetic improvement in swine comprising:
a. providing a first swine genetic nucleus elite breeding herd (hereinafter referred to as an “SGN”) at a first site effectively isolated from transmission of selected pathogens from a second site at which is located a SGN2 derived from the SGN1, the SGN1 having a rate of genetic improvement and a rate of inbreeding and a number of animals sufficient for achieving and maintaining over multiple generations a stable balance between the rate of genetic improvement and the rate of inbreeding, the SGN2 having a smaller number of animals than the SGN1, and the SGN1 and SGN2 being further genetically linked by use of semen from the same sires in producing offspring in both the SGN1 and SGN2;
b. generating a ranking of dams in the SGN2 for achieving a measure of genetic improvement for a next succeeding generation in the SGN2, optionally a target measure of improvement;
c. providing semen from sires in the SGN1 selected for use in breeding dams in the SGN2 to achieve the measure of genetic improvement in the SGN2; and
d. wherein the actual measure of genetic improvement in the SGN2 is determined at intervals and used for effecting genetic improvement in the SGN2.

2. The Method of claim 1 wherein the SGN1 and the SGN2 comprise SGNs for producing maternal line parent dams enriched in maternal traits for producing terminal swine.

3. The Method of claim 1 wherein offspring from the SGN1 and the SGN2 are evaluated for at least a core set of phenotypic data and the core set is used for generating the ranking of dams in the SGN2.

4. The Method of claim 1 wherein dams from the SGN2 are bred using semen from other lines, optionally from sires maintained in a boar stud herd at the second site, for producing via one or more crossbreeding steps, parent dams for producing terminal swine.

5. The Method of claim 1 wherein the core set of phenotypic data comprises, for the SGN1 and SGN2, reproductive data, off-test weight, and carcass data.

6. The Method of claim 1 wherein the core set of phenotypic data comprises, for the SGN2, reproductive data and off-test weight.

7. The Method of claim 1 wherein the core set of phenotypic data comprises, for the SGN2, only reproductive data.

8. The Method of claim 1 wherein the step of generating the ranking comprises use of a best linear unbiased prediction (BLUP) algorithm.

9. The Method of claim 1 wherein a dam herd for the SGN1 comprises more than about 450 animals.

10. The Method of claim 1 wherein a dam herd for the SGN2 comprises at least about 50 animals.

11. A method for producing genetic improvement in swine comprising:
a. maintaining, relative to a first swine genetic nucleus elite breeding herd (hereinafter referred to as an “SGN”) located at a first site effectively isolated from transmission of selected pathogens, a second site at which is located a SGN2 derived from the SGN1, the SGN1 having a rate of genetic improvement and a rate of inbreeding and a number of animals sufficient for achieving and maintaining over multiple generations a stable balance between the rate of genetic improvement and the rate of inbreeding, the SGN2 having a smaller number of animals than the SGN1, and the first and SGN2s being further genetically linked by use of semen from the same sires in producing offspring in both the first and SGN2s;
e. selecting dams for breeding from a ranking of dams in the SGN2 for achieving a measure of genetic improvement for a next succeeding generation in the SGN2, optionally a targeted measure of genetic improvement; and
b. at intervals evaluating the measure of genetic improvement and using the measure for breeding the selected dams using semen from sires in the SGN1 selected for use in breeding dams in the SGN2 to achieve the measure of genetic improvement in the SGN2.

12. The Method of claim 11 wherein the SGN1 and the SGN2 comprise SGNs for producing maternal line parent dams enriched in maternal traits for producing terminal swine.

13. The Method of claim 11 wherein offspring from the SGN1 and the SGN2 are evaluated for at least a core set of phenotypic data and the core set is used for generating the ranking of dams in the SGN2.

14. The Method of claim 11 wherein dams from the SGN2 are bred using semen from other lines, optionally from sires maintained in a boar stud herds at the second site, for producing via one or more crossbreeding steps, parent dams for producing terminal swine.

15. The Method of claim 11 wherein the core set of phenotypic data comprises, for the first and SGN2s, reproductive data, off-test weight, and carcass data.

16. The Method of claim 11 wherein the core set of phenotypic data comprises, for the SGN2, reproductive data and off-test weight.

17. The Method of claim 11 wherein the core set of phenotypic data comprises, for the SGN2, only reproductive data.

18. The Method of claim 11 wherein the step of generating the ranking comprises use of a best linear unbiased prediction (BLUP) algorithm.

19. The Method of claim 11 wherein a dam herd for the SGN 1 comprises more than about 450 animals and a sire line for the SGN1 comprises more than about 200 animals.

20. The Method of claim 11 wherein a dam herd for the SGN2 comprises at least about 50 animals.

21. A Method for determining measures for breeding swine comprising:
a. accessing at least a core set of phenotypic data obtained from each of a first swine genetic nucleus breeding herd SGN1 and a second swine genetic nucleus herd SGN2, the SGN1 and the SGN2 being genetically linked; and
b. producing measures for at least one of the SGN1 and the SGN2 herds selected from the group consisting of measures of estimated breeding values for selected traits and measures of rate of genetic improvement and combinations thereof.

22. The Method of claim 21 comprising:
a. producing measures for each of the SGN1 and SGN2 herds.

23. The Method of claim 21 comprising:
a. further accessing at least a core set of phenotypic data from each of an additional set of swine genetic nucleus breeding herds, each SGN genetically linked with at least one other of a resulting total set of swine genetic nucleus breeding herds SGNs; and
b. producing measures for at least one of the resulting total set of SGNs.

24. The Method of claim 21 wherein:
a. the measures of estimated breeding values are determined using a best linear unbiased prediction (BLUP) algorithm.

25. The Method of claim 21 wherein:
a. phenotypic data from at least one of SGN1 and SGN2 are provided by a data link to a database that is data linked to a data processor for producing the measures of estimated breeding values.

26. The Method of claim 21 further comprising:
a. Producing a measure of rate of genetic improvement for at least one of SGN1 and SGN2.

27. The Method of claim 21 further comprising:
a. Producing a measure of rate of genetic improvement for at least one of SGN1 and SGN2; and
b. Providing the measure of genetic improvement via a data link to a site associated with the swine genetics breeding herd for which the measure is produced.

28. The Method of claim 21 comprising:
a. Producing a measure of rate of genetic improvement for at least one of SGN1 and SGN2; and
b. Providing the measure of genetic improvement via a data link to a site associated with the swine genetics breeding herd for which the measure is produced;
c. Wherein the steps of producing and providing are conducted at multiple optionally regular intervals; and
d. Wherein the site receiving the measures uses the provided measures for measuring compliance with a predetermined breeding plan for the swine genetics nucleus herd associated with that site.

29. The Method of claim 28 comprising:
a. Improving compliance with the breeding plan upon occurrence of a provided rate of genetic improvement differing from a target rate of genetic improvement associated with the breeding plan.

30. The Method of claim 28 comprising:
a. Adjusting a target rate of genetic improvement associated with the breeding plan upon occurrence of a provided rate of genetic improvement differing from the target rate.

31. A method of producing genetic improvement in swine comprising:
a. accessing at least a core set of phenotypic data obtained from each of a first swine genetic nucleus breeding herd SGN1 and a second swine genetic nucleus herd SGN2, the SGN1 and the SGN2 being genetically linked; and
b. producing measures for the SGN2 herd selected from the group consisting of measures of estimated breeding values for selected traits and measures of rate of genetic improvement and combinations thereof;
f. generating a ranking of dams in the SGN2 herd for achieving a targeted measure of genetic improvement for a next succeeding generation in the SGN2 herd; and
g. providing the ranking of dams in SGN2 herd for use for selection of dams for breeding using semen from SGN1 selected for use in breeding dams in the SGN2 to achieve the targeted measure of genetic improvement in the SGN2.
